(19) United States
(12) Patent Application Publication
(10) Pub. No.: US 2020/0171194 A1
(43) Pub. Date: Jun. 4, 2020

(54) SCENT SWITCH DEVICE

(71) Applicant: SYMRISE AG, Holzminden (DE)

(72) Inventors: Jörg DRÖGE, Bodenwerder (DE); Ranjita SHEGOKAR, Goch (DE); Jörn WIEDEMANN, Holzminden (DE); Julia VON HOLLY, Holzminden (DE)

(21) Appl. No.: 16/636,386

(22) PCT Filed: Aug. 4, 2017
(86) PCT No.: PCT/EP2017/069813

§ 371 Date: Feb. 4, 2020

(51) Int. Cl. A61L 9/12; C11D 1/14; C11D 1/22; C11D 1/72; C11D 1/66; C11D 17/04; A61L 9/05; E03D 9/03

(52) U.S. Cl. CPC A61L 9/125; C11D 1/146; C11D 1/22; C11D 1/72; E03D 2009/024; C11D 17/046; A61L 9/05; E03D 9/032; A61L 2209/134; C11D 1/662

(57) ABSTRACT

The present invention relates to a device releasing substances in a controllable and changing way, particularly the present invention relates to the configuration of such a device (1) and the inner portion (2) of such device and to the use of such device to provide the release of the substances to the surrounding environment in a targeted way.

### TECHNICAL FIELD

The present invention relates to a device, preferably a device suitable for sanitary or air refreshing purposes, comprising an inner portion, which further comprises at least two compartments, each containing at least one different substance to provide a changing fragrance experience based on the targeted release of the substances in the at least two compartments of the inner portion based on the configuration of the device and based on the configuration the position of the compartment within the device.

### BACKGROUND

Sanitary and air deodorant devices for a variety of environments are relevant tools used in private and public rooms. The main purpose of said devices is to scavenge malodors and/or to positively influence the mood of a subject as mediated by a fragrance component comprised within the device.

One example for a sanitary device is a rim block. Typical rim blocks usually have to fulfill, disinfecting, cleaning and deodorizing purposes, and are composed of solid mass, gels or combinations of the same and a device for holding and fixing the active ingredient.

Currently rim blocks are available in a variety of cages which can deliver exclusively one fragrance with or without a cleaning agent. The disadvantage of this type of rim block is that consumers can experience only one fragrance all the time. The human and the animal brain and thus their perception has the tendency to shut the smell receptors after a few breaths and initial recognition of a fragrance, i.e., to get adapted to a fragrance experience, or to get used to said experience due to molecular mechanisms based on the relevant receptors. Therefore, within only a few breaths, the intensity of a smell provided fades and a subject or consumer cannot enjoy the fragrance over a longer period of time.

Furthermore, typical rim blocks swell inside their holders, when getting in contact with the flush water. Thus, the typical rim blocks lose their shape and are dissipated unevenly. Further, the holders are usually completely filled with flush water getting in contact with the rim block inside, which leads to an excessive dissipation of the rim block and thus potentially to an unpleasant consumer experience.

Within the last years, aesthetics and performance were of increasing importance and gelatinous or liquid rim blocks, possibly in rim blocks with several chambers, have been developed. However, still in the current rim blocks with several chambers, all chambers are having contact with water and the substances of all chambers are released simultaneously with every use. Still, only one fragrance, or substance, or mixture of substances, will be released by the device and will be experienced by the consumer and the consumer will always have the experience of the same substances when using such devices, which may lead to a possible nose blindness of the consumer. Further, with e.g., dual phase rim blocks, one phase tends to dissolve faster than the other phase, leading to a disparity in the dosing of the ingredients in the final flushes of the toilet as all rim block chambers are always equally accessible to flush water.

There is thus a great need in defining new rim block forms, to overcome scent related limitations by using a second generation device. Furthermore, specially designed fragrances can provide enhanced consumer experience, which may be suitable for a variety of further consumer product areas, in particular as a scent or fragrance component may be individually combined, e.g. with a suitable cleaning agent to achieve a dual fragrance, cleaning and/or disinfecting effect.

Moreover, new rim block forms may overcome an uneven and/or excessive dissipation of the rim block, by providing a targeted dissipation.

The object of the present invention was therefore to provide a device which releases changing substances to maintain the consumer's pleasure in the substance experience for a long period of time and in a targeted way to provide a varying fragrance experience over time.

### SUMMARY OF THE INVENTION

The above object is achieved by providing a device which is configured to allow the release of more than one fragrance substance comprised in different compartments based on the configuration of the device and its subcomponents, i.e., a device being configured for scent switch applications.

According to a first aspect of the present invention, the above object is achieved by providing a device (1) comprising an inner portion (2) attached to a suspension (3) and one or more rotation control devices (4) and optionally an outer portion (5), wherein the inner portion (2) comprises at least two compartments, each containing one or more substance(s) and being separated from each other by walls, and wherein the inner (2) and/or the optional outer (5) portion comprises at least one notch (6) configured to assure that only one defined compartment of the inner portion (2) at a time gets in contact with water and/or air, and wherein the, one, two or more or all rotation control device(s) (4) enable(s) rotation of the inner (2) and/or the optional outer (5) portion to assure that the rotation of the inner (2) and/or the optional outer (5) portion is stopped and/or activated to guarantee a targeted contact of water and/or air with one defined compartment of the inner portion (2).

In one embodiment, the, one, two or more or all of the substance(s) of at least one of the compartments can be in a solid, liquid or gas state, in a manner independent of any other of the substances.

In one embodiment, the, one, two or more or all of the substance(s) of at least one of the compartments may comprise at least one ingredient independently selected from the group consisting of
a. a fragrance and/or
b. a substance with anti-malodour activity and/or
c. a cleaning agent, such as sodium laureth sulfate, sodium dodecylbenzene sulfonate, fatty alcohol ethoxylate, oxoalcohol C13-15 ethoxylated, sodium lauryl sulphate or alkyl polyglycoside, an acid cleaner, and/or
d. a foam former, such as sodium laureth sulfate, sodium dodecylbenzene sulfonate, fatty alcohol ethoxylate, oxoalcohol C13-15 ethoxylated, sodium lauryl sulphate or alkyl polyglycoside, and/or
e. a chalk preventer, such as acids preferably being selected from the group consisting of citric acid, amidosulfonic acid, acetic acid, phosphoric acid, hydrochloric acid or lactic acid, and/or
f. a complexing agent, such as tetrasodium EDTA, diethylenetriamine pentamethylene phosphonic acid sodium salt,tri-sodiumcitrate, methylglycinediacetic acid, etidronic acid, a polyphosphate or a phosphonate and/or
g. an oxidative agent, such as a peroxide or a hypochloride, and/or
h. an antibacterial agent, such as an alcohol, chlorine, hydrogen peroxide, a quaternary ammonium compound, sodium hypochlorite, chlorine dioxide, a chloramine, a copper or silver ion, or colloidal silver, or bromine; and/or
i. any mixture or combination of the above at least one ingredient.

In another embodiment, the device (1) comprises an outer portion (5), and the notch (6) may be at least one window in the wall of the outer portion (5), which has a size not bigger than one compartment of the inner portion (2), and the outer portion (5) may have one or more opening(s) (7).

In one embodiment, the device (1) comprises an outer portion (5), and the, one, two or more or all rotation control device(s) (4) may assure that only one compartment is covered by the notch (6), and the one or more substance(s) of any one of the at least two compartments can be solid.

In another embodiment, the notch (6) can be at least one window in the wall of any one of the at least two compartments of the inner portion (2), and the inner portion (2) may comprise at least one closing flap (8) for each window, and the one or more substance(s) of the at least two compartments can be in a gas form or liquid.

In one embodiment, the, one, two or more or all rotation control device(s) (4) can be independently selected from the group consisting of a disc, a rotor, a valve, a mechanical stopper, a flap, a hatch, an angular controller, a ring, a joint, a socket, a lock and key model or any combination thereof.

In another embodiment, the, one, two or more or all rotation control device(s) (4) may be configured to stop the rotation of the inner (2) and/or outer (5) portion at a position where only one defined compartment of the inner portion (2) at a time gets in contact with water and/or air, and said defined compartment can be a different one as the compartment before the rotation.

In another embodiment, the device (1) comprises an outer portion (5), and the outer portion (5) may comprise one or more opening(s) (7), wherein the one or more opening(s) (7) can be fine pores.

In one embodiment, the one or more rotation control device(s) (4) can be positioned
a. proximal to the inner portion (2), and/or
b. distal to the inner portion (2), and/or
c. inside the inner portion (2).

In another embodiment, the device (1) comprises an outer portion (5), and the one or more rotation control device(s) (4) may be positioned
a. proximal to the inner portion (2) and (a1) inside or (a2) outside the outer portion (5), and/or
b. distal to the inner portion (2) and (b1) inside or (b2) outside the outer portion (5), and/or
c. inside the inner portion (2).

In another embodiment, any one of the inner portion (2), the suspension (3), the at least one rotation control device (4), the outer portion (5) or the at least one closing flap (8), if present, can be made of at least one material selected from the group consisting of
a. a synthetic material, such as polyethylene terephthalate (PET), polyethylene terephthalate-G (PETG), polyvinyl chloride (PVC), polypropylene (PP), polystyrene (PS), high impact polystyrene (HIPS), polyvinylidene chloride (PVDC), high-density polyethylene (HDPE) or low-density polyethylene (LDPE) and/or
b. a metal, such as copper, aluminium, rust free iron, steel, and/or
c. a magnetic or magnetisable material, and/or
d. silicon, cardboard, eva beads, and/or
e. a glass, porcelain or ceramic substance and/or
f. wood or cork and/or
g. any combination of the above at least one material.

In one embodiment, the device (1) can be a sanitary device, preferably a toilet rim block, a (room) air modifying device, such as an air refreshing device, for example an air refreshing device for use in closed rooms, or a device for use within another device, such as in an oven, a cupboard or a fridge, or device for use in open areas, or an air refreshing device for use in a transportation vehicle, for example a car, train, or aircraft.

A second aspect of the present invention comprises the inner portion (2) as defined above, wherein all of the compartments of the inner portion (2) can be constructed
a. to be refillable, or
b. not to be refillable.

A further aspect of the present invention comprises the use of a device (1) and/or the use of an inner portion (2), both as defined above, for providing the release of at least one ingredient selected from the group consisting of
a. a fragrance and/or
b. a substance with anti-malodour activity and/or
c. a cleaning agent, such as sodium laureth sulfate, sodium dodecylbenzene sulfonate, fatty alcohol ethoxylate, oxoalcohol C13-15 ethoxylated, sodium lauryl sulphate or alkyl polyglycoside, an acid cleaner, and/or
d. a foam former, such as sodium laureth sulfate, sodium dodecylbenzene sulfonate, fatty alcohol ethoxylate, oxoalcohol C13-15 ethoxylated, sodium lauryl sulphate or alkyl polyglycoside, and/or
e. a chalk preventer, such as acids preferably being selected from the group consisting of citric acid, amidosulfonic acid, acetic acid, phosphoric acid, hydrochloric acid or lactic acid, and/or
f. a complexing agent, such as tetrasodium EDTA, diethylenetriamine pentamethylene phosphonic acid sodium salt,tri-sodiumcitrate, methylglycinediacetic acid, etidronic acid, a polyphosphate or a phosphonate and/or
g. an oxidative agent, such as a peroxide or a hypochloride, and/or
h. an antibacterial agent, such as an alcohol, chlorine, hydrogen peroxide, a quaternary ammonium compound, sodium hypochlorite, chlorine dioxide, a chloramine, a copper or silver ion, or colloidal silver, or bromine; and/or
i. any mixture or combination of the above at least one ingredient
   to the surrounding environment in a targeted way, preferably to modify or mask malodour in a variety of environments, and/or to modify the mood of a subject in the surrounding environment in a targeted way.

### BRIEF DESCRIPTION OF THE DRAWINGS

FIG. 1 is a diagonal view of one embodiment of the device (1) according to the invention, comprising an inner portion (2), a suspension (3), an outer portion (5) with a notch (6) and openings (7).

FIG. 2 is a diagonal view of one embodiment of the device (1) according to the invention, not comprising an outer portion (5), but an inner portion (2), a suspension (3) a notch (6) and closing flaps (8) and at least one liquid substance in the compartments of the inner portion (2).

FIG. 3 is a diagonal view of an exemplary rotation control device (4) according to the invention, further comprising stop mechanisms (4a).

FIG. 4 is a cross-sectional view of an exemplary inner portion (2), consisting of two (left), three (middle) or four (right) compartments.

### DETAILED DESCRIPTION

The present invention thus provides a variety of different configurations for devices suitable for scent switch applications.

According to a first aspect of the present invention, there is provided a device (1) comprising an inner portion (2) attached to a suspension (3) and one or more rotation control devices (4) and optionally an outer portion (5), wherein the inner portion (2) comprises at least two compartments, each containing one or more substance(s) and being separated from each other by walls, and wherein the inner (2) and/or the optional outer (5) portion comprises at least one notch (6) configured to assure that only one defined compartment of the inner portion (2) at a time gets in contact with water and/or air, and wherein the, one, two or more or all rotation control device(s) (4) enable(s) rotation of the inner (2) and/or the optional outer (5) portion to assure that the rotation of the inner (2) and/or the optional outer (5) portion is stopped and/or activated to guarantee a targeted contact of water and/or air with one defined compartment of the inner portion (2).

The device according to the invention thus provides a facilitated use to the consumer. The consumer is provided with a multiple substance containing device, thus allowing him to apply a plurality of substances in a targeted and controlled way, with only one device.

Furthermore, the device according to the invention improves the consumer's perception of the substances contained in such device. As the inner (2) and/or the optional outer (5) portion of the device (1) rotate and only one defined compartment of the inner portion (2) may get in contact with water and/or air, the substances released from the device (1) are differing. Therefore, the consumer's adaptation to a fragrance or to an experience of a substance contained in the device (1) is reduced.

In one embodiment according to the present invention, the shape of the inner (2) and optional outer (5) portion may be cylindrical, spherical, cubic, pyramidal, cuneiform or wedge-shaped, in spinner form, or in the form of a thin sheet. Usually, the shape of the inner (2) and optional outer (5) portion will be adapted to the environment where the device is used. For air refreshing devices being used in a detached form, there will thus be fewer limitations than for installed or attachable devices, e.g., for rim blocks or devices being used as filter cartridges in a car for providing a scent switch device.

In one embodiment of the present invention, the at least two compartments of the inner portion (2) are separated from each other by walls, wherein the walls can be made of any material suitable for preventing a diffusion of any of the substance(s) from one compartment to the other, or for preventing any other mixture of the substances of different compartments. If the device comprises an outer portion (5), the sides of the compartments of the inner portion (2) facing the outer portion (5) and/or the notch (6) may also be uncovered, partially uncovered and/or may also have a notch (6) as described above.

In one embodiment of the present invention, the suspension (3) is constructed in a way that the inner (2) and/or optional outer (5) portion are attached to the suspension (3) but can rotate along the axis of the suspension (3).

In a further embodiment according to the present invention, the at least one notch (6) is configured in a shape, a size, and if more than one, a distribution, that guarantees that only one compartment at a time is covered by the at least one notch (6). The rotation control device(s) (4) further position the inner (2) and/or the optional outer (5) portion in a way, that only one compartment of the inner portion (2) is covered by the one or all notch(es) (6) at a given time.

In one embodiment according to the present invention, the compartments of the inner portion (2) may further be subdivided in sub-compartments, which contain one or more substance(s) that may differ from those of other sub-compartments. All aspects of the present invention that apply for a compartment also apply for a compartment being subdivided.

Water and/or air of the environment surrounding the device (1) is/are intended to flow through the device (1). Optionally, the rotation of the inner (2) and/or the optional outer (5) portion may be thereby driven by the flow of water and/or air.

In yet a further embodiment of the present invention, the, one, two or more or all rotation control device(s) (4) may stop and/or activate the rotation of the inner (2) and/or the optional outer (5) portion.

For stopping the rotation, the, one, two or more or all rotation control device(s) (4) may comprise a stop mechanism (4a), which ensures a stop of the rotation in determined positions, thereby positioning the inner (2) and/or optional outer (5) portion in a way that only one defined compartment of the inner portion (2) at a time gets in contact with water and/or air. Such stop mechanisms (4a) may be selected from the group of spatial barriers, magnetic stoppers, wooden, plastic or metal disks, hatches, rings, weight controlled mechanisms, a position detecting mechanism, gravitational mechanisms, rotating mechanism, or flaps. For activating the rotation, the, one, two or more or all rotation control device(s) (4) may possess a structure for activating rotation, such as propellers, paddles, wooden, plastic or metal disks, hatches, rings, weight controlled mechanisms, a position detecting mechanism, gravitational mechanisms, rotating mechanism or electric mechanisms, which can be propelled by the water and/or air of the surrounding environment, by an electric motor, by the air pressure in the surrounding environment, manually, gravitation or a piezo crystal, or any combination thereof.

In one embodiment, the activating mechanism can comprise at least one sensor, for example a sensor for light, movement or a person sitting down on the toilet, or the activating mechanism can comprise a timer for activating the rotation of the inner (2) and/or optional outer (5) portion.

Furthermore, water and/or air is/are intended to interact with the at least one substance of the respective compartment of the inner portion (2) by establishing a targeted contact with the at least one substance.

A targeted contact of water and/or air with one defined compartment of the inner portion (2) comprises a contact of water and/or air with the substance(s) contained within the respective compartment of the inner portion (2). Such a contact can be made possible by the at least one notch (6) of the inner (2) and/or the optional outer (5) portion.

A targeted contact further comprises the contact of water and/or air with only one of the compartments of the inner portion (2), and the substance(s) within, at a time. Such a contact can be temporally limited. The temporally limited contact has the advantage that less material is consumed as there is exclusively one interacting compartment at a time. This results in a decreased consumption of the at least one substance in the at least two compartments and thus results in a more ecological and sustainable device, at the same time having the changing user experience over time. Therefore, the device according to the present invention is configured as multifunctional device which may have individually and/or synergistically acting components with different effects such as a perfuming, anti-malodour, cleaning, foam forming, chalk protection, antibacterial, disinfecting, oxidating or complexing effect. Thus, the device has the advantages of being user friendly as it can be refilled manually and with individually selected substances. Furthermore, the device combines additional economic advantages, as less waste is produced than with single used devices. These economic factors are combined with a greatly enhanced physical comfort on the side of the consumer.

A notch (6) in the outer portion (5) may enable water and/or air from outside the outer portion (5) entering the outer portion (5) and getting in physical contact with the substance(s) contained within the respective compartment of the inner portion (2).

A notch (6) in the inner portion (2) may provide the release of the substance(s) contained within the respective compartment of the inner portion (2) from the respective compartment of the inner portion (2) and thus enables physical contact of water and/or air with the respective substance(s). Further, a notch (6) in the inner portion (2) may enable water and/or air from outside the inner (2) and/or optional outer portion (5) entering the inner portion (2) and getting in physical contact with the substance(s) contained within the respective compartment of the inner portion (2).

A targeted contact of water and/or air with the substance(s) contained within the respective compartment of the inner portion (2) is meant to comprise the uptake of a portion of the respective substance(s) into water and/or air, resulting in a solution, dispersion or mixture of substance(s) with water and/or air. Such a solution, dispersion or mixture is meant to leave or be released from the device (1) so that the at least one ingredient, preferably at least one fragrance, comprised can be released and thus be perceived by the consumer.

A device according to the invention thus has further advantages by providing a targeted contact of water and/or air with the substance(s) contained within the respective compartment of the inner portion (2). Due to the configuration of the compartments of the inner portion (2), only the outer surface of the substance may be in contact and interact with the water and/or air. The dissipation of the substance within the compartment is much lower, compared with typical devices of the state of the art, where the whole substance is washed round. Thus, an excessive dissipation is avoided and a device according to the invention is thus more sustainable and economical than typical devices of the state of the art.

For free-standing devices, in particular air refreshing devices, to be placed in closed rooms, within devices, such as an oven, a cupboard or a fridge, or within devices used in open areas, or within an air refreshing device for use in a transportation vehicle, for example, a car, train, or aircraft, the rotation control device (4) and the notch (6) can be optional. Furthermore, in certain embodiments the rotation of the inner (2) and/or outer (5) portion to each other may not be necessary for allowing controlled access to the at least two compartments.

In one embodiment of the invention, the, one, two or more of the substance(s) of at least one of the compartments may be in a solid, liquid, or gas state, in a manner independent of any other of the substances. The state of the substance may largely depend on the usage environment of the device and the purpose of the device.

The term liquid is meant to be understood as a state of a substance in which a flux of the substance is possible. Such a state comprises the further conditions low, medium and highly viscous, paste-like and the like.

The term solid is meant to be understood as a state of a substance in which a flux of the substance is not possible and the term further comprises the state semisolid, gelatinous and the like.

According to the various aspects of the present invention, the, one, two or more of the substance(s) of at least one of the compartments may comprise at least one ingredient independently selected from the group consisting of
a. a fragrance and/or
b. a substance with anti-malodour activity and/or
c. a cleaning agent, such as sodium laureth sulfate, sodium dodecylbenzene sulfonate, fatty alcohol ethoxylate, oxoalcohol C13-15 ethoxylated, sodium lauryl sulphate or alkyl polyglycoside, an acid cleaner, and/or
d. a foam former, such as sodium laureth sulfate, sodium dodecylbenzene sulfonate, fatty alcohol ethoxylate, oxoalcohol C13-15 ethoxylated, sodium lauryl sulphate or alkyl polyglycoside, and/or
e. a chalk preventer, such as acids preferably being selected from the group consisting of citric acid, amidosulfonic acid, acetic acid, phosphoric acid, hydrochloric acid or lactic acid, and/or
f. a complexing agent, such as tetrasodium EDTA, diethylenetriamine pentamethylene phosphonic acid sodium salt,tri-sodiumcitrate, methylglycinediacetic acid, etidronic acid, a polyphosphate or a phosphonate and/or
g. an oxidative agent, such as a peroxide or a hypochloride, and/or
h. an antibacterial agent, such as an alcohol, chlorine, hydrogen peroxide, a quaternary ammonium compound, sodium hypochlorite, chlorine dioxide, a chloramine, a copper or a silver ion, or colloidal silver, or bromine; and/or
i. any mixture or combination of the above at least one ingredient.

Fragrances are meant to be understood as fragrances selected from the group consisting of
Anethole, benzaldehyde, benzyl acetate, benzyl alcohol, benzyl formate, iso-bornyl acetate, camphene, ciscitral (neral), citronellal, citronellol, citronellyl acetate, p-cymene, decanal, dihydrolinalool, dihydromyrcenol, dimethyl phenyl carbinol, eucaliptol, geranial, geraniol, geranyl acetate, geranyl nitrile, cis-3-hexenyl acetate, hydroxycitronellal, d-limonene, linalool, linalool oxide, linalyl acetate, linalyl propionate, methyl anthranilate, alpha-methyl ionone, methyl nonyl acetaldehyde, methyl phenyl carbinyl acetate, laevo-menthyl acetate, menthone, iso-menthone, mycrene, myrcenyl acetate, myrcenol, nerol, neryl acetate, nonyl acetate, phenyl ethyl alcohol, alpha-pinene, beta-pinene, gamma-terpinene, alpha-terpineol, beta-terpineol, terpinyl acetate, and vertenex (p-tertiary-butyl cyclohexyl acetate), amyl cinnamic aldehyde, isoamyl salicylate, beta-caryophyllene, cedrene, cinnamic alcohol, coumarin, dimethyl benzyl carbinyl acetate, ethyl vanillin, eugenol, iso-eugenol, flor acetate, heliotropine, 3-cis-hexenyl salicylate, hexyl salicylate, filial (p-tertiarybutyl-alpha-methyl hydrocinnamic aldehyde), gamma-methyl ionone, nerolidol, patchouli alcohol, phenyl hexanol, beta-selinene, trichloromethyl phenyl carbinyl acetate, triethyl citrate, vanillin, and veratraldehyde. Cedarwood terpenes are composed mainly of alpha-cedrene, beta-cedrene, and other C15H24 sesquiterpenes, benzophenone, benzyl salicylate, ethylene brassylate, galaxolide (1,3,4,6,7,8-hexahydro-4,6,6,7,8,8-hexamethyl-cyclopenta-gama-2-benzopyran), hexyl cinnamic aldehyde, lyral (4-(4-hydroxy-4- methyl pentyl)-3- cyclohexene- 10-carboxaldehyde), methyl cedrylone, methyl dihydro jasmonate, methyl-beta-naphthyl ketone, musk indanone, musk ketone, musk tibetene, and phenylethyl phenyl acetate, angelica root oil, anise oil, arnica flower oil, basil oil, bay oil, champaca flower oil, silver fir oil, silver fir cone oil, elemi oil, eucalyptus oil, fennel oil, spruce needle oil, galbanum oil, geranium oil, ginger grass oil, guaiacwood oil, gurjun balsam oil, helichrysum oil, ho oil, ginger oil, iris oil, cajeput oil, calamus oil, chamomile oil, camphor oil, cananga oil, cardamom oil, cassia oil, pine needle oil, copaiba balsam oil, coriander oil, spearmint oil, caraway oil, cumin oil, lemongrass oil, musk seed oil, myrrh oil, clove oil, neroli oil, niaouli oil, olibanum oil, oregano oil, palmarosa oil, patchouli oil, Peru balsam oil, petitgrain oil, pepper oil, peppermint oil, pimento oil, pine oil, rosemary oil, sandalwood oil, celery oil, star anise oil, thuja oil, thyme oil, verbena oil, vetiver oil, juniper berry oil, wormwood oil, wintergreen oil, ylang-ylang oil, hyssop oil, cinnamon oil, cinnamon leaf oil and cypress oil, ambrettolide, α-amyl cinnamaldehyde, anethole, anisaldehyde, anisyl alcohol, anisole, anthranilic acid methyl ester, acetophenone, benzyl acetone, benzaldehyde, benzoic acid ethyl ester, benzophenone, benzyl alcohol, borneol, bornyl acetate, α-bromostyrene, n-decyl aldehyde, n-dodecyl aldehyde, eugenol, eugenol methyl ether, eucalyptol, farnesol, fenchone, fenchyl acetate, geranyl acetate, geranyl formate, heliotropin, heptyne carboxylic acid methyl ester, heptaldehyde, hydroquinone dimethyl ether, hydroxycinnamaldehyde, hydroxycinnamyl alcohol, indole, irone, isoeugenol, isoeugenol methyl ether, isosafrole, jasmone, camphor, carvacrol, carvone, p-cresol methyl ether, coumarin, p-methoxyacetophenone, methyl-n-amyl ketone, methylanthranilic acid methyl ester, p-methyl acetophenone, methyl chavicol, p-methyl quinoline, methyl-β-naphthyl ketone, methyl-n-nonyl acetaldehyde, methyl-n-nonyl ketone, muscone, β-naphthol ethyl ether, β-naphthol methyl ether, nerol, nitrobenzene, n-nonyl aldehyde, nonyl alcohol, n-octyl aldehyde, p-oxyacetophenone, pentadecanolide, β-phenylethyl alcohol, phenylacetaldehyde dimethyl acetal, phenylacetic acid, pulegone, safrole, salicylic acid isoamyl ester, salicylic acid methyl ester, salicylic acid hexyl ester, salicylic acid cyclohexyl ester, santalol, skatole, terpineol, thymene, thymol, y-undecalactone, vanillin, veratrum aldehyde, cinnamaldehyde, cinnamyl alcohol, cinnamic acid, cinnamic acid ethyl ester and cinnamic acid benzyl ester, alkyl isothiocyanates (alkyl mustard oils), buta-nedione, limonene, linalool, linalyl acetate, linalyl propionate, menthol, menthone, methyl-n-heptenone, phellandrene, phenylacetaldehyde, terpinyl acetate, citral and citronellal;
C8-C8 hydrocarbons: delta-3-carene, alpha-pinene, beta-pinene, alpha-terpinene, gamma-terpinene, p-cymene, bisabolene, camphene, caryophyllene, cedrene, farnesene, limonene, longifolene, myrcene, ocimene, valencene, (E,Z)-1,3,5-undecatriene);
C2-C8 aliphatic alcohols: hexanol, octanol, 3-octanol, 2,6-dimethylheptanol, 2-methylheptanol, 2-methyloctanol, (E)-3-hexenol, (E) and (Z)-3-hexenol, 1-octen-3-ol, mixtures of 3,4,5, 6,6-pentamethyl-3/4-hepten-2-ol and 3,5,6,6-tetramethyl-4-methyleneheptan-2-ol, (E,Z)-2,6-nonadienol, 3,7-dimethyl-7-methoxyoctan-2-ol, 9-decenol, 10-undecenol, 4-methyl-3-decen-5-ol;
C2-C8 aliphatic aldehydes and their acetals: hexanal, heptanal, octanal, nonanal, decanal, unde-canal, dodecanal, tridecanal, 2-methyloctanal, 2-methylnonanal, (E)-2-hexenal, (Z)-4-heptenal, 2,6-dimethyl-5-heptenal, 10-undecenal, (E)-4-decenal, 2-dodecenal, 2,6, 10-trimethyl-5,9-undecadienal, heptanal diethyl acetal, 1,1-dimethoxy-2,2,5-trimethyl-4-hexene, citronellyl oxyacetaldehyde;
C3-C8 aliphatic ketones and oximes thereof: 2-heptanone, 2-octanone, 3-octanone, 2-nonanone, 5-methyl-3-heptanone, 5-methyl-3-heptanone oxime, 2,4,4,7-tetramethyl-6-octen-3-one;
C2-C8 aliphatic sulphur-containing compounds: 3-methylthiohexanol, 3-methylthiohexyl acetate, 3-mercaptohexanol, 3-mercaptohexyl acetate, 3-mercaptohexyl butyrate, 3-acetylthiohexyl acetate, 1-menthene-8-thiol;
C2-C8 aliphatic nitrile-containing compounds: 2-nonenenitrile, 2-tridecenenenitrile, 2,12-tridecenene-nitrile, 3,7-dimethyl-2,6-octadienenitrile, 3,7-dimethyl-6-octenenitrile.
C2-C18 aliphatic carboxylic acids and esters thereof: (E)-and (Z)-3-hexenyl formate, ethyl acetoacetate, isoamyl acetate, hexyl acetate, 3,5,5-trimethylhexyl acetate, 3-methyl-2-butenyl acetate, (E)-2-hexenyl acetate, (E)-and (Z)-3-hexenyl acetate, octyl acetate, 3-octyl acetate, 1-octen-3-yl acetate, ethyl butyrate, butyl butyrate, isoamyl butyrate, hexyl butyrate, (E)-and (Z)-3-hexenyl isobutyrate, hexyl crotonate, ethyl isovalerate, ethyl 2-methylpentanoate, ethyl hexanoate, allyl hexanoate, ethyl heptanoate, allyl heptanoate, ethyl octanoate, ethyl (E,Z)-2,4-decadienoate, methyl 2-octynoate, methyl 2-nonynoate, allyl-2-isoamyloxyacetate, methyl-3,7-dimethyl-2,6-octadienoate;
C4-C18 acyclic terpene alcohols: citronellol, geraniol, nerol, linalool, lavandulol, nerolidol, farnesol, tetrahydrolinalool, tetrahydrogeraniol, 2,6-dimethyl-7-octen-2-ol, 2,6-dimethyloctan-2-ol, 2-methyl-6-methylene-7-octen-2-ol, 2,6-dimethyl-5,7-octadien-2-ol, 2,6-dimethyl-3,5-octadien-2-ol, 3,7-dimethyl-4,6-octadien-3-ol, 3,7-dimethyl-1,5,7-octatrien-3-ol, 2,6-dimethyl-2,5,7-octatrien-1-ol;
C4-C8 acyclic terpene aldehydes and ketones: geranial, neral, citronellal, 7-hydroxy-3,7-dimethyloctanal, 7-m ethoxy-3,7-dimethyloctanal, 2,6,10-trimethyl-9-undecenal, geranylace-tone, and the dimethyl and diethyl acetals of geranial, neral, 7-hydroxy-3,7-dimethyloctanal;
C4-C8 cyclic terpene alcohols: alpha-terpineol, terpineol-4, menthan-8-ol, menthan-1-ol, menthan-7-ol, borneol, isoborneol, linalool oxide, nopol, cedrol, ambrinol, vetiverol, guaiol;
C4-C8 cyclic terpene aldehydes and ketones: fenchone, alpha-ionone, beta-ionone, alpha-n-methylionone, beta-n-methylionone, alpha-isomethylionone, beta-isomethylionone, alpha-irone, alpha-damascone, beta-damascone, beta-damascenone, delta-damascone, gamma-damascone, 1-(2,4,4-trimethyl-2-cyclohexen-1-yl)-2-buten-1-one, 1,3,4,6,7,8a-hexahydro-1,1,5,5-tetramethyl-2H-2,4a-methanonaphthalen-8(5H)-one, nootkatone, dihydronootkatone, alpha-sinensal, beta-sinensal, methyl cedryl ketone;
C4-C8 cyclic alcohols: 4-tert-butylcyclohexanol, 3,3,5-trimethylcyclohexanol, 3-isocamphylcyclohexanol, 2,6,9-trimethyl-Z2,Z5,E9-cyclododecatrien-1-ol, 2-isobutyl-4-methyltetrahydro-2H-pyran-4-ol);
C4-C8 cycloaliphatic alcohols: alpha-3,3-trimethylcyclohexylmethanol, 2-methyl-4-(2,2,3-trimethyl-3-cyclopent-1-yl)butanol, 2-methyl-4-(2,2,3-trimethyl-3-cyclopent-1-yl)-2-buten1l-ol, 2-ethyl-4-(2,2,3-trimethyl-3-cyclopent-1-yl)-2-buten-1-ol, 3-methyl-5-(2,2,3-trimethyl-3-cyclopent-1-yl)-pentan-2-ol, 3-methyl-5-(2,2,3-trimethyl-3-cyclopent-1-yl)-4-penten-2-ol, 3,3-dimethyl-5-(2,2,3-trimethyl-3-cyclopent-1-yl)-4-penten-2-ol, 1-(2,2,6-trimethylcyclohexyl)pentan-3-ol, 1-(2,2,6-trimethylcyclohexyl)hexan-3-ol;
C4-C8 cyclic and cycloaliphatic ethers: cedryl methyl ether, cyclododecyl methyl ether, (ethoxymeth-oxy)cyclododecane, alpha-cedrene epoxide, 3a,6,6,9a-tetramethyl-dodecahydronaphtho[2,1-b]furan, 3a-ethyl-6,6,9a-trimethyldodecahydro-naphtho[2,1-b]furan, 1,5,9-trimethyl-13-oxabicyclo[10.1.0]trideca-4,8-diene, rose oxide, 2-(2,4-dimethyl-3-cyclohexen-1-yl)-5-methyl-5-(1-methylpropyl)-1,3-dioxane);
C4-C8 cyclic ketones: 4-tert-butylcyclohexanone, 2,2,5-trimethyl-5-pentylcyclopentanone, 2-heptylcyclopentanone, 2-pentylcyclopentanone, 2-hydroxy-3-methyl-2-cyclopenten-1-one, 3-methyl-cis-2-penten-1-yl-2-cyclopenten-1-one, 3-methyl-2-pentyl-2-cyclopenten-1-one, 3-methyl-4-cyclopentadecenone, 3-methyl-5-cyclopentadecenone,3-methylcyclopentadecanone, 4-(1-ethoxyvinyl)-3,3,5,5-tetramethylcyclohexanone, 4-tert-pentylcyclohexanone, 5-cyclohexadecen-1-one, 6,7-dihydro-1, 1,2,3, 3-pentamethyl-4(5H)-indanone, 9-cycloheptadecen-1-one, cyclopenta-decanone, cyclohexadecanone;
C4-C18 cycloaliphatic aldehydes: 2,4-dimethyl-3-cyclohexenecarbaldehyde, 2-methyl-4-(2,2,6-trimethyl-cyclohexen-1-yl)-2-butenal, 4-(4-hydroxy-4-methylpentyl)-3-cyclohexenecarbaldehyde, 4-(4-methyl-3-penten-1-yl)-3-cyclohexenecarbaldehyde;
C4-C18 cycloaliphatic ketones: 1-(3,3-dimethylcyclohexyl)-4-penten-1-one, dimethyl-1-cyclohexen-1-yl)-4-penten-1-one, 2,3,8,8-tetramethyl-1,2,3,4,5,6,7,8-octahydro-2-naphthalenyl methyl ketone, methyl-2,6,10-trimethyl-2,5,9-cyclododecatrienyl ketone, tert-butyl(2,4-dimethyl-3-cyclohexen-1-yl)ketone);
esters of cyclic alcohols in C4-C18: 2-tert-butylcyclohexyl acetate, 4-tert-butyl-cyclohexyl acetate, 2-tert-pentylcyclohexyl acetate, 4-tert-pentylcyclohexyl acetate, decahy-dro-2-naphthyl acetate, 3-pentyltetrahydro-2H-pyran-4-yl acetate, decahydro-2,5,5,8a-tetramethyl-2-naphthyl acetate, 4,7-methano-3a,4,5,6,7,7a-hexahydro-5 or 6-indenyl acetate, 4,7-methano-3a,4,5,6,7,7a-hexahydro-5 or 6-indenyl propionate, 4,7-methano-3a,4,5,6,7,7a-hexahydro-5 or 6-indenyl isobutyrate, 4,7-methanooctahydro-5 or 6-indenyl acetate);
esters of cycloaliphatic carboxylic acids in C4-C8: allyl 3-cyclohexylpropionate, allyl cyclohexyloxyacetate, methyl dihydrojasmonate, methyl jasmonate, methyl 2-hexyl-3-oxocyclopentanecarboxylate, ethyl 2-ethyl-6,6-dimethyl-2-cyclohexenecarboxylate, ethyl 2,3,6,6-tetramethyl-2-cyclohexenecarboxylate, ethyl 2-methyl-1,3-d ioxolane-2-acetate;
C4-C8 aromatic hydrocarbons: styrene and diphenylmethane;
C4-C18 araliphatic alcohols: Benzyl alcohol, 1-phenylethyl alcohol, 2-phenylethyl alcohol, 3-phenylpropanol, 2-phenylpropanol, 2-phenoxyethanol, 2,2-dimethyl-3-phenylpropanol, 2,2-dimethyl-3-(3-methylphenyl)propanol, 1,1-dimethyl-2-phenylethyl alcohol, 1,1-dimethyl-3-phenylpropanol, 1-ethyl-1-methyl-3-phenylpropanol, 2-methyl-5-phenylpentanol, 3-methyl-5-phenylpentanol, 3-phenyl-2-propen-1-ol, 4-methoxybenzyl alcohol, 1-(4-isopropylphenyl)ethanol);
esters of araliphatic alcohols in C4-C18 and aliphatic carboxylic acids in C4-C18:

benzyl acetate, benzyl propionate, benzyl isobutyrate, benzyl isovalerate, 2-phenylethyl acetate, 2-phenylethyl propionate, 2-phenylethyl isobutyrate, 2-phenylethyl isovalerate, 1-phenylethyl acetate, alpha-trichloromethylbenzyl acetate, alpha,alpha-dimethylphenylethyl acetate, alpha,alpha-dimethylphenylethyl butyrate, cinnamyl acetate, 2-phenoxyethyl isobutyrate, 4-methoxybenzyl acetate); C2-C18 araliphatic ethers (such as 2-phenylethyl methyl ether, 2-phenylethyl isoamyl ether, 2-phenylethyl-1-ethoxyethyl ether, phenylacetaldehyde dimethyl acetal, phenylacetaldehyde diethyl acetal, hydratropaldehyde dimethyl acetal, phenylacetal-dehyde glycerol acetal, 2,4,6-trimethyl-4-phenyl-1,3-dioxane, 4,4a,5,9b-tetrahydroindeno[1,2-d]m-dioxin, 4,4a,5,9b-tetrahydro-2,4-dimethylindeno[1,2-d]-m-dioxin;
C4-C18 aromatic and araliphatic aldehydes: benzaldehyde, phenylacetaldehyde, 3-phenylpropanal, hydratropaldehyde, 4-methylbenzaldehyde, 4-methylphenylacetaldehyde, 3-(4-ethylphenyl)-2,2-dimethylpropanal, 2-methyl-3-(4-isopropylphenyl)propanal, 2-methyl-3-(4-tert.-butylphenyl)propanal, 3-(4-tert-butylphenyl)propanal, cinnamaldehyde, alpha-butylcinnamaldehyde, alpha-amylcinnamaldehyde, alpha-hexylcinnamaldehyde, 3-methyl-5-phenylpentanal, 4-methoxybenzaldehyde, 4-hydroxy-3-methoxybenzaldehyde, 4-hydroxy-3-ethoxybenzaldehyde, 3,4-methylenedioxybenzaldehyde, 3,4-dimethoxybenzaldehyde, 2-m ethyl-3-(4-methoxyphenyl)propanal, 2-methyl-3-(4-methylenedioxyphenyl)propanal;
C4-C18 aromatic and araliphatic ketones: acetophenone, 4-methylacetophenone, 4-methoxyacetophenone, 4-tert-butyl-2,6-dimethylacetophenone, 4-phenyl-2-butanone, 4-(4-hydroxyphenyl)-2-butanone, 1-(2-naphthalenyl)ethanone, benzophenone, 1,1,2,3,3,6-hexamethyl-5-indanyl methyl ketone, 6-tert-butyl-1,1-dimethyl-4-indanyl methyl ketone, 1-[2,3-dihydro-1,1,2,6-tetramethyl-3-(1-methylethyl)-1H-5-indenyl]ethanone, 5′,6′,7′,8′-tetrahydro-3′,5′,5′,6′,8′,8′-hexamethyl-2-acetonaphthone);
C4-C18 aromatic and araliphatic carboxylic acids and esters thereof: phenylacetic acid, methyl benzoate, ethyl benzoate, hexyl benzoate, benzyl benzoate, methyl phenylacetate, ethyl phenylacetate, geranyl phenylacetate, phenylethyl phenylacetate, methyl cinnamate, ethyl cinnamate, benzyl cinnamate, phenylethyl cinnamate, cinnamyl cinnamate, allyl phenoxyacetate, methyl salicylate, isoamyl salicylate, hexyl salicylate, cyclohexyl salicylate, cis-3-hexenyl salicylate, benzyl salicylate, phenylethyl salicylate, methyl 2,4-dihydroxy-3,6-dimethylbenzoate, ethyl 3-phenylglycidate, ethyl 3-methyl-3-phenylglycidate); nitrogen-containing aromatic compounds in C4-C18 (such as 2,4,6-trinitro-1,3-dimethyl-5-tert-butylbenzene, 3,5-dinitro-2,6-dimethyl-4-tert-butylacetophenone, cinna-monitrile, 5-phenyl-3-methyl-2-pentenenitrile, 5-phenyl-3-methylpentanenitrile, methyl anthranilate, methyl N-methylanthranilate, Schiff bases of methyl anthranilate with 7-hydroxy-3,7-dimethyloctanal, 2-methyl-3-(4-tert-butylphenyl)propanal, 2,4-dimethyl-3-cyclohexene-carbaldehyde, 6-isopropylquinoline, 6-isobutylquinoline, 6-sec-butylquinoline, indole, skatole, 2-methoxy-3-isopropylpyrazine, 2-isobutyl-3-methoxypyrazine); phenols, phenyl ethers and phenyl esters (such as estragole, anethole, eugenol, eugenyl methyl ether, isoeugenol, isoeugenyl methyl ether, thymol, carvacrol, diphenyl ether, beta-naphthyl methyl ether, beta-naphthyl ethyl ether, beta-naphthyl isobutyl ether, 1,4-dimethoxybenzene, eugenyl acetate, 2-methoxy-4-methylphenol, 2-ethoxy-5-(1-propenyl)phenol, p-cresyl phenylacetate;
hetero-cyclic compounds in C4-C12: 2,5-dimethyl-4-hydroxy-2H-furan-3-one, 2-ethyl-4-hydroxy-5-methyl-2H-furan-3-one, 3-hydroxy-2-methyl-4H-pyran-4-one, 2-ethyl-3-hydroxy-4H-pyran-4-one);
lactones in C4-C12: 1,4-octanolide, 3-methyl-1,4-octanolide, 1,4-nonanolide, 1,4-decanolide, 8-decen-1,4-olide, 1,4-undecanolide, 1,4-dodecanolide, 1,5-decanolide, 1,5-dodecanolide, 1,15-pentadecanolide, cis- and trans-pentadecen-1,15-olide, cis-and trans-12-pentadecen-1,15-olide, 1,16-hexadecanolide, 9-hexadecen-1,16-olide, 10-oxa-1,16-hexadecanolide, 11-oxa-1, 16-hexadecanolide, 12-oxa-1,16-hexadecanolide, ethylene 1,12-dodecanedioate, ethylene 1, 13-tridecanedioate, coumarin, 2,3-dihydrocoumarin, octahydrocoumarin.
Further fragrances are available to the skilled person and can be used according to the present invention alone or in combination.

A substance with anti-malodour activity is meant to be understood as a substance specially designed to neutralize, reduce, mask and/or prevent malodours. Such substances may comprise tailor-made perfume oils to overcome specific environmental, base- or body-related malodours. Substances with anti-malodour activity can be selected from the group of:
Substances according to the Neofresh® technology;
Esters: 2,4-dimethyl-3-pentyl propionate, 2,4-dimethyl-3-pentyl isobutyrate, 2,4-dimethyl-3-pentyl crotonate, 2,4-dimethyl-3-pentyl butyrate, 2,6-dimethyl-4-heptyl propionate, 2,6-dimethyl-4-heptyl isobutyrate, 2,6-dimethyl-4-heptyl crotonate, 2,6-dimethyl-4-heptyl butyrate, 3,3,5-trimethylcyclohexyl acetate, 3,3,5-trimethylcyclohexyl propionate, 3,3,5-trimethylcyclohexyl crotonate, 3,3,5-trimethylcyclohexyl butyrate, menthyl propionate, menthyl isobutyrate, menthyl crotonate, menthyl butyrate, isomenthyl acetate, isomenthyl propionate, isomenthyl isobutyrate, isomenthyl crotonate, isomenthyl butyrate, isopulegyl propionate, isopulegyl isobutyrate, isopulegyl crotonate, isopulegyl butyrate, 2,6,6-trimethylcycloheptyl acetate, 2,6,6-trimethylcycloheptyl propionate, 2,6,6-trimethylcycloheptyl isobutyrate, 2,6,6-trimethylcycloheptyl crotonate, 2,6,6-trimethylcycloheptyl butyrate, 2,2,4-trimethyl-1,3-pentanediyl diacetate, 2,2,4-trimethyl-1,3-pentanediyl dipropionate, 2,2,4-trimethyl-1,3-pentanediyl diisobutyrate, 2,2,4-trimethyl-1,3-pentanediyl dicrotonate, 2,2,4-trimethyl-1,3-pentanediyl dibutyrate, 2-methyl-2 ,4-pentanediyl diacetate, 2-methyl-2,4-pentanediyl dipropionate, 2-methyl-2,4-pentanediyl diisobutyrate, 2-methyl-2,4-pentanediyl dicrotonate, 2-methyl-2,4-pentanediyl dibutyrate. 2,4-dimethyl-3-pentyl propionate, 2,4-dimethyl-3-pentyl crotonate, 2,4-dimethyl-3-pentyl butyrate, 2,6-dimethyl-4-heptyl propionate, 2,6-dimethyl-4-heptyl isobutyrate, 2,6-dimethyl-4-heptyl crotonate, 2,6-dimethyl-4-heptyl butyrate, isomenthyl isobutyrate, isomenthyl crotonate, isomenthyl butyrate, isopulegyl crotonate, 2-methyl-2,4-pentanediyl dicrotonate and 2.2.4-trimethyl-1,3-pentanediyl dicrotonate;
Volatile aldehydes: Adoxal (2,6,10-trimethyl-9-undecenal), bourgeonal (4-t-butylbenzenepropionaldehyde), lilestralis 33 (2-methyl-4-t-butylphenyl)propanal), cinnamic aldehyde, cinnamaldehyde (phenyl propenel, 3-phenyl-2-propenal), citral, geranial, neral (dimethyloctadienal, 3,7-dimethyl-2,6-octadien-1-al), cyclal c (2,4-dimethyl-3-cyclohexen-1-carbaldehyde), florhydral (3-(3-isopropyl-phenyl)-butyraldehyde), citronellal (3,7-dimethyl 6-octenal), cymal, cyclamen aldehyde, cyclosal, lime aldehyde (alpha-methyl-p-isopropyl phenyl propyl aldehyde), methyl nonyl acetaldehyde, aldehyde c12 mna (2-methyl-1-undecanal), hydroxycitronellal, citronellal hydrate (7-hydroxy-3,7-dimethyl octan-1-al), helional (alpha-methyl-3,4-(methylenedioxy)-hydrocinnamaldehyde, hydrocinnamaldehyde (3-phenylpropanal, 3-phenylpropionaldehyde), intreleven aldehyde (undec-10-en-1-al), ligustral, trivertal (2,4-dimethyl-3-cyclohexene-1- carboxaldehyde), jasmorange, satinaldehyde (2-methyl-3-tolylproionaldehyde, 4-dimethylbenzenepropanal), lyral (4-(4-hydroxy-4-methyl pentyl)-3-cyclohexene-1-carboxaldehyde), melonal (2,6-dimethyl-5-heptenal), methoxy melonal (6-methoxy-2,6-dimethylheptanal), methoxycinnamaldehyde (trans-4-methoxycinnamaldehyde), myrac aldehyde isohexenyl cyclohexenyl-carboxaldehyde, trifernal ((3-methyl-4-phenyl propanal, 3-phenyl butanal), filial, p.t. bucinal, lysmeral, benzenepropanal (4-tert-butyl-alpha-methyl-hydrocinnamaldehyde), du pical, tricyclodecylidenebutanal (4-triyclo5210-2,6decylidene-8butanal), melafleur (1,2,3,4,5,6,7,8-octahydro-8,8-dimethyl-2-naphthaldehyde), methyl octyl acetaldehyde, aldehyde c-11 moa (2-methyl deca-1-al), onicidal (2,6,10-trimethyl-5,9-undecadien-1-al), citronellyl oxyacetaldehyde, muguet aldehyde 50 (3,7-dimethyl-6-octenyl) oxyacetaldehyde), phenylacetaldehyde, mefranal (3-methyl-5-phenyl pentanal), triplal, vertocitral dimethyl tetrahydrobenzene aldehyde (2,4-dimethyl-3-cyclohexene-1-carboxaldehyde), 2-phenylproprionaldehyde, hydrotropaldehyde, canthoxal, anisylpropanal 4-methoxy-alpha-methyl benzenepropanal (2-anisylidene propanal), cylcemone a (1,2,3,4,5,6,7,8-octahydro-8,8-dimethyl-2-naphthaldehyde), and precylcemone b (1-cyclohexene-1-carboxaldehyde), acetaldehyde (ethanal), pentanal, valeraldehyde, amylaldehyde, scentenal (octahydro-5-methoxy-4,7-methano-1h-indene-2-carboxaldehyde), propionaldehyde (propanal), cyclocitral, beta-cyclocitral, (2,6,6-trimethyl-1-cyclohexene-1-acetaldehyde), iso cyclocitral (2,4 ,6-trimethyl-3-cyclohexene-1-carboxaldehyde), isobutyraldehyde, butyraldehyde, isovaleraldehyde (3-methyl butyraldehyde), methylbutyraldehyde (2-methyl butyraldehyde, 2-methyl butanal), dihydrocitronellal (3,7-dimethyl octan-1-al), 2-ethylbutyraldehyde, 3-methyl-2-butenal, 2-methylpentanal, 2-methyl valeraldehyde, hexenal (2-hexenal, trans-2-hexenal), heptanal, octanal, nonanal, decanal, lauric aldehyde, tridecanal, 2-dodecanal, methylthiobutanal, glutaraldehyde, pentanedial, glutaric aldehyde, heptenal, cis or trans-heptenal, undecenal (2-, 10-), 2,4-octadienal, nonenal (2-, 6-), decenal (2-, 4-), 2,4-hexadienal, 2,4-decadienal, 2,6-nonadienal, octenal, 2,6-dimethyl 5-heptenal, 2-isopropyl-5-methyl-2-hexenal, trifernal, beta methyl benzenepropanal, 2,6,6-trimethyl-1-cyclohexene-1-acetaldehyde, phenyl butenal (2-phenyl 2-butenal), 2.methyl-3(p-isopropylphenyl)-propionaldehyde, 3-(p-isopropylphenyl)-propionaldehyde, p-tolylacetaldehyde (4-methylphenylacetaldehyde), anisaldehyde (p-methoxybenzene aldehyde), benzaldehyde, vernaldehyde (1-methyl-4-(4-methylpentyl)-3-cyclohexenecarbaldehyde), heliotropin (piperonal) 3,4-methylene dioxy benzaldehyde, alpha-amylcinnamic aldehyde, 2-pentyl-3-phenylpropenoic aldehyde, vanillin (4-methoxy 3-hydroxy benzaldehyde), ethyl vanillin (3-ethoxy 4-hydroxybenzaldehyde), hexyl cinnamic aldehyde, jasmonal h (alpha-n-hexyl-cinnamaldehyde), floralozone, (para-ethyl-alpha,alpha-dimethyl hydrocinnamaldehyde), acalea (p-methyl-alpha-pentylcinnamaldehyde), methylcinnamaldehyde, alpha-methylcinnamaldehyde (2-methyl 3-pheny propenal), alpha-hexylcinnamaldehyde (2-hexyl 3-phenyl propenal), salicylaldehyde (2-hydroxy benzaldehyde), 4-ethyl benzaldehyde, cuminaldehyde (4-isopropyl benzaldehyde), ethoxybenzaldehyde, 2,4-dimethylbenzaldehyde, veratraldehyde (3,4-dimethoxybenzaldehyde), syringaldehyde (3,5-dimethoxy 4-hydroxybenzaldehyde), catechaldehyde (3,4-dihydroxybenzaldehyde), safranal (2,6,6-trimethyl-1,3-diene methanal), myrtenal (pin-2-ene-1-carbaldehyde), perillaldehydel-4(1-methylethenyl)-1-cyclohexene-1-methylpentenal, carboxaldehyde), 2,4-dimethyl-3-cyclohexene carboxaldehyde, 2-methyl-2-pentenal, 2-pyruvaldehyde, formyl tricyclodecan, mandarin aldehyde, cyclemax, pino acetaldehyde, corps iris, maceal, and corps 4322;
Acid catalysts: Formic acid, acetic acid, trimethyl acetic acid, phenol (alkaline in liquid apps yet acidic in vapor phase), tiglic acid, caprylic acid, 5-methyl thiophene carboxylic acid, succinic acid, benzoic acid, mesitylenic acid;
Malodour absorbers: Molecular sieves, such as zeolites, silicas, aluminosilcates, and cyclodextrins; and organic absorbents, such as for example, activated charcoal, dried citrus pulp, cherry pit extract, corncob.

An acid cleaner is meant to be understood as acids with a cleaning function to be used according to the present invention. Acid cleaners may comprise:
Organic acids: Formic acid, acetic acid, citric acid, glycolic acid, lactic acid, succinic acid, adipic acid, malic acid, tartaric acid and gluconic acid.
Inorganic acids: Hydrochloric acid, sulfuric acid, phosphoric acid and nitric acid or sulfamic acid.

The at least one substance of at least one of the compartments may further comprise ingredients such as:

### Cleaning Agents:

Surfactants:
Nonionic surfactant: Fatty alcohol alkoxylate, such as alkoxylated C12-C20 fatty alcohols, or alkoxylated C16-C18 fatty alcohols, ethylene oxide/propylene oxide block polymers, fatty acid alkanolamides, fatty acid polyglycol ethers, alkyl polyglycosides, fatty acid glucamides, aliphatic sulfates, such as fatty alcohol sulfates, fatty alcohol ether sulfates, dialkyl ether sulfates, monoglyceride sulfates, and aliphatic sulfonates, such as alkane sulfonates, ether sulfonates, n-alkyl ether sulfonates, ester sulfonates and lignin sulfonates, fatty acid cyanamides, sulfosuccinates (sulfosuccinic acid esters), such as sulfosuccinic acid mono- and di-C8-C18-alkyl esters, sulfosuccinamates, sulfosucci-namides, fatty acid isethionates, acylaminoalkane sulfonates (fatty acid taurides), fatty acid sarcosinates, ether carboxylic acids and alkyl (ether) phosphates as well as α-sulfo fatty acid salts, acyl glutamates, monoglyceride disulfates and alkyl ethers of glycerol disulfate.
Anionic surfactants: Alkyl benzene sulfonate, C8-C18 alkyl sulphate, alkyl ether sulfates, sulfonates such as the alkyl sulfonates, the olefin sulfonates, the alkyloxy, the alkyl aryl sulfonates, sulfate esters, the alkyl carbonates, alkyl ether carboxylates, the fatty acid taurides, isethionates, soaps, alkyl benzenesulfonates, alkanesulfonates, olefin sulfonates, alkylether sulfonates, glycerol ether sulfonates, methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oli-goglucoside sulfates, protein fatty acid condensates (particularly wheat-based vegetable products) and alkyl (ether) phosphate.
   Complexing agents:
Aminotrimethylene phosphonic acid, beta-alanine diacetic acid, calcium disodium EDTA, citric acid, cyclodextrin, cyclohexanediamine tetraacetic acid, diammonium citrate, diammonium EDTA, diethylenetriamine pentamethylene phosphonic acid, dipotassium EDTA, disodium azacycloheptane diphosphonate, disodium EDTA, disodium pyrophosphate, EDTA, etidronic acid, galactaric acid, gluconic acid, glucuronic acid, HEDTA, hydroxypropyl cyclodextrin, methyl cyclodextrin, pentapotassium triphosphate, pentasodium aminotrimethylene phosphonate, pentasodium ethylenediamine tetramethylene phosphonate, pentasodium pentetate, pentasodium triphosphate, pentetic acid, phytic acid, Potassium citrate, potassium EDTMP, potassium gluconate, potassium polyphosphate, potassium trisphosphonomethylamine oxide, ribonic acid, sodium chitosan methylene phosphonate, sodium citrate, sodium diethylenetriamine pentamethylene phosphonate, sodium dihydroxyethylglycinate, sodium EDTMP, sodium gluceptate, sodium gluconate, sodium glycereth-1 polyphosphate, sodium hexametaphosphate, sodium metaphosphate, sodium metasilicate, sodium phytate, sodium polydimethylglycinophenolsulfonate, sodium trimetaphosphate, TEA-EDTA, TEA-polyphosphate, tetrahydroxyethyl ethylenediamine, tetrahydroxypropyl ethylenediamine, tetrapotassium etidronate, tetrapotassium pyrophosphate, tetrasodium EDTA, tetrasodium etidronate, tetrasodium pyrophosphate, tripotassium EDTA, trisodium dicarboxymethyl alaninate, risodium EDTA, trisodium HEDTA, trisodium NTA and risodium phosphate.

### Biocides:

Amine oxides, CTAC (cetyl trimethyl ammonium chloride), and BAC (benzalkonium chloride), metal salts such as zinc citrate, zinc oxide, zinc pyrethiones, and octopirox; organic acids, such as sorbic acid, benzoic acid, and their salts; parabens, such as methyl paraben, propyl paraben, butyl paraben, ethyl paraben, isopropyl paraben, isobutyl paraben, benzyl paraben, and their salts; alcohols, such as benzyl alcohol, phenyl ethyl alcohol; boric acid; 2,4,4′- trichloro-2-hydroxy-diphenyl ether; phenolic compounds, such as phenol, 2-methyl phenol, 4-ethyl phenol; essential oils such as rosemary, thyme, lavender, eugenol, geranium, tea tree, clove, lemon grass, peppermint, or their active components such as anethole, thymol, eucalyptol, farnesol, menthol, limonene, methyl salicylate, salicylic acid, terpineol, nerolidol, geraniol, ethanol, n-propanol, i-propanol, 1, 3-butanediol, phenoxyethanol, 1, 2-propylene glycol, glycerol, undecylenic acid, citric acid, lactic acid, benzoic acid, salicylic acid, thymol, 2-benzyl-4-chlorophenol, 2,2′-methylene-bis-(6-bromo-4-chloro phenol), 2,4,4′-trichloro-2′-hydroxydiphenyl ether, N-(4-chlorophenyl)-N-(3,4-dichlorophenyl)- urea, N, N (1, 10-decandiyldi-1-pyridinyl-4 ylidene)-bis-(1-octanamine) dihydrochloride, N, N′-bis (4-chlorophenyl)-3,12-diimino-2,4, 1 1, 13-tetraazatetradecanediimidamide, antimicrobial surface-active quaternary compounds, guanidines and sodium dichloroisocyanurate (DCI, 1, 3-dichloro-5H-1, 3,5-triazine-2,4,6-trione sodium salt).

### Corrosion Inhibitors:

Cyclohexylamine, diammonium phosphate, dilithium oxalate, dimethylamino methylpropanol, dipotassium oxalate, dipotassium phosphate, disodium phosphate, disodium pyrophosphate, disodium tetrapropenyl succinate, hexoxyethyl diethylammonium, phosphate, nitromethane, potassium silicate, sodium aluminate, sodium hexametaphosphate, sodium metasilicate, sodium molybdate, sodium nitrite, sodium oxalate, sodium silicate, stearamidopropyl dimethicone, tetrapotassium pyrophosphate, tetrasodium pyrophosphate and triisopropanolamine.

### Enzymes:

Proteases, lipases, amylases, hydrolases and/or cellulases.

### Bleaching Agents:

Peroxides, per acids and/or perborates, hydrogen peroxide; sodium percarbonate or phthalimidoperoxyhexanoic acid.

The substance(s), leaving or being released from the device (1) as solution, dispersion or mixture with water and/or air, can advantageously fulfil specific functions especially desirable in the sanitary field such as providing good smell, being a support in cleaning and prevention of chalk and a combination of those. The functions are preferably fulfilled outside the device.

Any combination or mixture of the aforementioned substances can be perceived according to the present invention. A combination or mixture according to the invention will largely depend on the usage environment, i.e., the place, where the device is placed and thus used, and the usage purpose, i.e., sanitary purposes, air refreshing purposes and the like.

As the inner (2) and/or the optional outer (5) portion of the device (1) according to the present invention rotates and only one compartment of the inner portion (2) may be in contact with water and/or air, different substances can be released from the device (1). Therefore, functional substances that are not mandatory in each use of the device but may be required to be released e.g. only every second or third use, can be applied to the surrounding environment in an appropriate way. This is particularly suitable for expensive substances, where a controllable and economical release is desired, or for substances, for example harsh cleaning agents, which could destroy the surrounding environment, or which could cause bleaching if the respective substance is continuously released.

According to the various aspects of the present invention, the device (1) may comprise an outer portion (5), wherein the notch (6) is at least one window in the wall of the outer portion (5), which has a size not bigger than one compartment of the inner portion (2), and wherein the outer portion (5) may have one or more opening(s) (7).

Windows in the wall of the outer portion (5) are to be understood as gaps or other openings in the wall of the outer portion (5). Such openings enable the contact between the substance(s) of the compartments of the inner portion (2), which they are covering, and the environment outside the outer portion (5), which may comprise water and/or air and preferably collects a portion of the substance(s) of the inner portion (2) during contact, as described above.

The term at least one window, which has a size not bigger than one compartment, comprises one or more windows, wherein the area of the wall of the outer portion (5), which is covered by the one or more windows and on which the one or more windows are distributed over the wall of the outer portion (5), is not bigger than the surface of one compartment facing the outer portion. Moreover, the area of the wall of the outer portion (5), which is covered by the one or more window(s) and on which the one or more window(s) is/are distributed over the wall of the outer portion (5) has a shape and size equal to one compartment of the inner portion (2). Furthermore, the one or all window(s) is/are positioned on such a defined area. Thereby it is ensured that not more than one compartment of the inner portion (2) at a time gets in contact with water and/or air through such window(s).

If water enters the outer portion (5), it is advantageous if the outer portion (5) comprises one or more opening(s) (7), so that the water will not remain inside the outer portion (5), but can get out through the opening(s). By enabling the water to be removed from inside the outer portion (5), the risk of inside remaining water having contact with compartments of the inner portion (2) other than desired or inside remaining water serving as ground for fungi, bacteria or the like is reduced. Furthermore, undue interaction between water and a substance in a given compartment can be reduced so that the consumption of the at least one substance can be kept low in an economic way.

In an embodiment, comprising an outer portion (5), the, one, two or more or all rotation control device(s) (4) may assure that only one compartment of the inner portion (2) is covered by the notch (6), and the one or more substance(s) of any one of the at least two compartments may be solid.

In an embodiment, not comprising an outer portion (5), the notch (6) may be at least one window in the wall of any one of the at least two compartments of the inner portion (2), the inner portion (2) may comprise at least one closing flap (8) for each window, and the one or more substance(s) of the at least two compartments may be in a gas form or liquid.

The at least one closing flap (8) may be opened and closed due to gravitational force: The closing flap (8) of the compartment at the bottom of the device (1) is pulled down by gravitational force and uncovers its window, whereas the closing flap of the compartment at the top of the device (1) is pushed down by gravitational force and covers its window (FIG. 2). As a result of rotation of the inner (2) and/or the optional outer (5) portion, the open and close state of the respective compartments is changed.

If the substance(s) of the compartment of the inner portion (2) comprise(s) a liquid, the notch (6) may comprise further constrictions, valves, or the like to ensure a controlled release of the liquid, e.g. a viscous liquid, a gel, or a suspension.

If the substance(s) of the compartment of the inner portion (2) comprise(s) a gas, the open and close mechanism may not be controlled only by gravity but by a mechanical and/or magnetic and/or electric mechanism ensuring a defined temporal limitation of the opening time of the notch (6).

In one embodiment, the at least one substance within one of the compartment of the inner portion (2) may be provided in a capsule form.

As an alternative of or addition to gravitational force controlling the opening time of the at least one window, the device (1) may comprise a mechanical and/or magnetic and/or electric mechanism and/or a clock regulating the opening times of the at least one window in determined time intervals.

According to the various aspects of the present invention, the, one, two or more or all rotation control device(s) (4) may be independently selected from the group consisting of a disc, a rotor, a valve, a mechanical stopper, a flap, a hatch, an angular controller, a ring, a joint, a socket, a lock and key model or any combination thereof.

According to the various aspects of the present invention, the, one, two or more or all rotation control device(s) (4) may be configured to stop the rotation of the inner (2) and/or optional outer (5) portion at a position where only one defined compartment of the inner portion (2) at a time gets in contact with water and/or air, and said defined compartment may be a different one as the compartment before the rotation.

In an embodiment of the invention, comprising an outer portion (5), the outer portion (5) may comprise one or more opening(s) (7), wherein the one or more opening(s) (7) may be fine pores.

The use of fine pores as one or more opening(s) (7) in the outer portion (5) is an advantage as the fine pores facilitate the removal of water from inside the outer portion (5) and simultaneously provide adequate protection of the compartments of the inner portion (2) not being positioned at the notch(es) (6) and optionally not covered at this side.

According to the various aspects of the present invention, the one or more rotation control device(s) (4) may be positioned
a. proximal to the inner portion (2), and/or
b. distal to the inner portion (2), and/or
c. inside the inner portion (2).

In another embodiment, comprising an outer portion (5), the one or more rotation control device(s) (4) may be positioned
a. proximal to the inner portion (2) and (a1) inside or (a2) outside the outer portion (5), and/or
b. distal to the inner portion (2) and (b1) inside or (b2) outside the outer portion (5), and/or
c. inside the inner portion (2).

According to the various aspects of the present invention, any one of the inner portion (2), the suspension (3), the at least one rotation control device (4), the outer portion (5) or the at least one closing flap (8), if present, may be made of at least one material selected from the group consisting of
a. a synthetic material, such as polyethylene terephthalate (PET), polyethylene terephthalate-G (PETG), polyvinyl chloride (PVC), polypropylene (PP), polystyrene (PS), high impact polystyrene (HIPS), polyvinylidene chloride (PVDC), high-density polyethylene (HDPE) or low-density polyethylene (LDPE) and/or
b. a metal, such as copper, aluminium, rust free iron, steel, and/or
c. a magnetic or magnetisable material and/or
d. silicon, cardboard, eva beads, and/or
e. a glass, porcelain or ceramic substance and/or
f. wood or cork and/or
g. any combination of the above at least one material.

According to the various aspects of the present invention, the device (1) may be a sanitary device, preferably a toilet rim block, a (room or open area) air modifying device, such as an air refreshing device, for example an air refreshing device for use in closed rooms, or a device for use within another device, such as in an oven, a cupboard or a fridge, or device for use in open areas, or an air refreshing device for use in a transportation vehicle, for example a car, train, or aircraft.

In another aspect of the present invention, all of the compartments of the inner portion (2), according to the present invention and its embodiments, may be constructed
a. to be refillable, or
b. not to be refillable, i.e., being configured for single use.

The inner portion (2) can be constructed in such a way, that, for refilling, the respective substance(s) may be added directly to the respective compartment of the inner portion (2) or as tanks, cartridges, blocks, capsules, pads, tabs or the like, wherein the inner portion (2) contains a holding device for tanks, cartridges, blocks, capsules, pads, tabs or the like.

Alternatively, the inner portion (2) can be constructed in such a way, that, if not refillable, the inner portion (2) can be exchanged as a whole.

To refill or exchange the inner portion (2), the outer portion (5), if present, is further constructed in a way that the inner portion (2) can be refilled or exchanged. Refilling or exchanging of the inner portion (2) can be enabled by an opening, opening mechanism or the like within the outer portion (5), suitable for refilling our exchanging the inner portion (2).

Another aspect of the present invention comprises the use of a device (1) according to the invention and its embodiments, and/or the use of an inner portion (2) according to the invention and its various embodiments, for providing the release of at least one ingredient selected from the group consisting of
a. a fragrance and/or
b. a substance with anti-malodour activity and/or
c. a cleaning agent, such as sodium laureth sulfate, sodium dodecylbenzene sulfonate, fatty alcohol ethoxylate, oxoalcohol C13-15 ethoxylated, sodium lauryl sulphate or alkyl polyglycoside, an acid cleaner, and/or
d. a foam former, such as sodium laureth sulfate, sodium dodecylbenzene sulfonate, fatty alcohol ethoxylate, oxoalcohol C13-15 ethoxylated, sodium lauryl sulphate or alkyl polyglycoside, and/or
e. a chalk preventer, such as acids preferably being selected from the group consisting of citric acid, amidosulfonic acid, acetic acid, phosphoric acid, hydrochloric acid or lactic acid, and/or
f. a complexing agent, such as tetrasodium EDTA, diethylenetriamine pentamethylene phosphonic acid sodium salt,tri-sodiumcitrate, methylglycinediacetic acid, etidronic acid, a polyphosphate or a phosphonate and/or
g. an oxidative agent, such as a peroxide or a hypochloride, and/or
h. an antibacterial agent, such as an alcohol, chlorine, hydrogen peroxide, a quaternary ammonium compound, sodium hypochlorite, chlorine dioxide, a chloramine, a copper or silver ion, or colloidal silver, or bromine; and/or
i. any mixture or combination of the above at least one ingredient
   to the surrounding environment in a targeted way, preferably to modify or mask malodour in a variety of environments, and/or to modify the mood of a subject in the surrounding environment in a targeted way.

The surrounding environment is to be understood as the environment around the device (1), in which the device (1) is intended to be used. Such an environment may comprise closed rooms, especially in houses or transportation vehicles, such as cars, trains, or aircrafts, or open areas.

The expressions “proximal to” and “distal to” the inner portion (2) are referred to the figures in an alignment such that the reference numbers and the notations of the figures can be read in normal orientation. “Proximal to” equals “left of” and “distal to” equals “right of”, referring to the figures. In the figures:

Preferred embodiments and further aspects of the present invention emerge from the attached patent claims, the figures and the following examples, the examples not being intended to limit the scope of the present invention.

### EXAMPLES

### Example 1

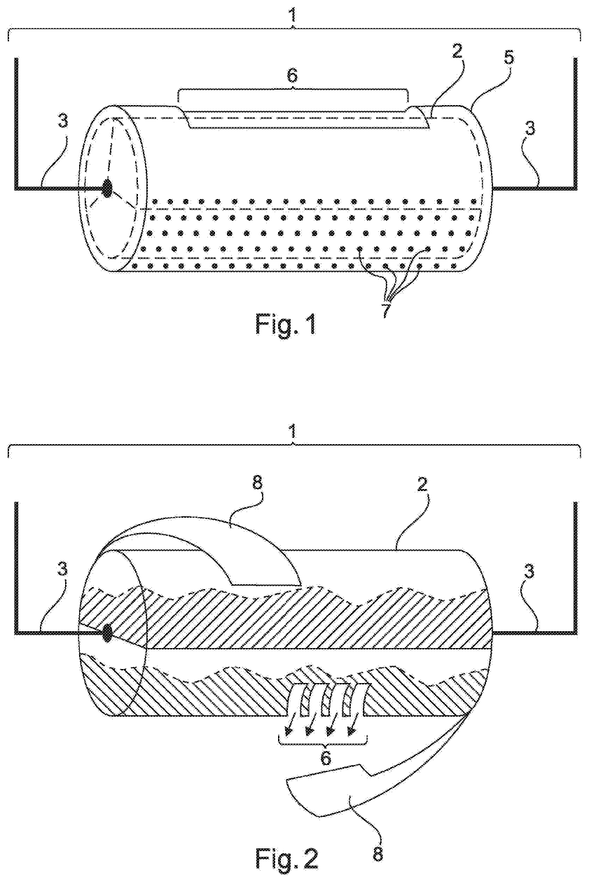

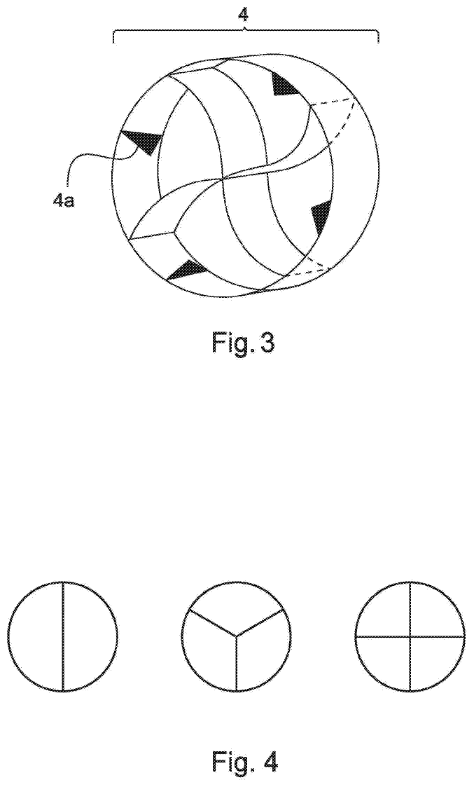

To provide a prototypic device for use in toilet purposes, the three dimension printing device “Ultimaker 2” was used. The device as shown in FIG. 1 was constructed using polylactic acid (PLA) and acrylonitrile butadiene styrene (ABS) as material. Other plastic/polymer materials were used to achieve a prototypic device having varying flexibility to test, which parts of the device have to be more rigid or more flexible. The various experiments aimed at providing a device which has low production costs, a high flexibility to be placed in a variety of different toilet bowls, and still a certain stiffness to allow the positioning of varying compartments inside the device. Further tests with different cleaning and fragrance compositions were done. For example, one rim block in a reusable configuration, and a one-way use rim block were printed. Notably, the material for reusable configurations should resist to certain mechanical movements so that the inner portion with the at least two or several different containers or compartments can be easily removed and replaced without damaging the device or the holder. For configurations of the device using a mechanical stopper (also used in a rotation control device as shown in FIG. 4) it turned out to be suitable to use a stiffer material for those parts being under higher mechanical stress to guarantee a certain longevity of the device and a proper movement mechanism.

### Example 2

Furthermore, it was tested whether scent switch devices could be used as air refreshing tools. A device was constructed, comprising three compartments. The change of the active compartment was initiated by a timer and performed by an electric motor. The stop mechanism of the rotation control device was a position detecting mechanism. Compartment 1 was filled with the fragrance geranial, compartment 2 was filled with the fragrance patchouli oil and compartment 3 was filled with floralozone, having anti-malodour activity. The device provided the changing substances to the surrounding environment and the fragrances and substances with anti-malodour activity were experienced for a longer time than with typical devices.

1. A device (1) comprising an inner portion (2) attached to a suspension (3) and one or more rotation control device(s) (4) and optionally an outer portion (5),
wherein the inner portion (2) comprises at least two compartments, each containing one or more substance(s) and being separated from each other by walls, and
wherein the inner (2) and/or the optional outer (5) portion comprises at least one notch (6) configured to assure that only one defined compartment of the inner portion (2) at a time gets in contact with water and/or air, and
wherein the one or more control device(s) (4) enables rotation of the inner (2) and/or the optional outer (5) portion to assure that the rotation of the inner (2) and/or the optional outer (5) portion is stopped and/or activated to guarantee a targeted contact of water and/or air with one defined compartment of the inner portion (2).

2. The device (1) according to claim 1, wherein the one or more substance(s) of at least one of the compartments is/are in a solid, liquid, or gas state, in a manner independent of any other of the substances.

3. The device (1) according to claim 1, wherein the one or more substance(s) of at least one of the compartments comprise(s) at least one ingredient independently selected from the group consisting of
a. a fragrance,
b. a substance with anti-malodour activity,
c. a cleaning agent,
d. a foam former,
e. a chalk preventer,
f. a complexing agent,
g. an oxidative agent,
h. an antibacterial agent, and
i. any mixture or combination of the above at least one ingredient.

4. The device (1) according to claim 1, wherein the device (1) comprises an outer portion (5), and wherein the notch (6) is at least one window in the wall of the outer portion (5), which has a size not bigger than one compartment of the inner portion (2).

5. The device (1) according to claim 4, wherein the one or more control device(s) (4) assure(s) that only one compartment is covered by the notch (6), and wherein the one or more substance(s) of any one of the at least two compartments is/are solid.

6. The device (1) according to claim 1, wherein the notch (6) is at least one window in the wall of any one of the at least two compartments of the inner portion (2), and wherein the inner portion (2) comprises at least one closing flap (8) for each window, and wherein the one or more substance(s) of the at least two compartments is/are in a gas form or liquid.

7. The device (1) according to claim 1, wherein the one or more control device(s) (4) is/are independently selected from the group consisting of a disc, a rotor, a valve, a mechanical stopper, a flap, a hatch, an angular controller, a ring, a joint, a socket, a lock and key model or any combination thereof.

8. The device (1) according to claim 1, wherein the one or more control device(s) (4) is/are configured to stop the rotation of the inner (2) and/or outer (5) portion at a position where only one defined compartment of the inner portion (2) at a time gets in contact with water and/or air, and wherein said defined compartment is a different one as the compartment before the rotation.

9. The device (1) according to claim 1, wherein the device (1) comprises an outer portion (5), and wherein the outer portion (5) comprises one or more opening(s) (7), wherein the one or more opening(s) (7) is/are fine pores.

10. The device (1) according to claim 1, wherein the one or more rotation control device(s) (4) is/are positioned
a. proximal to the inner portion (2),
b. distal to the inner portion (2), or
c. inside the inner portion (2).

11. The device (1) according to claim 1, wherein the device (1) comprises an outer portion (5), and wherein the one or more rotation control device(s) (4) is/are positioned
a. proximal to the inner portion (2) and (a1) inside or (a2) outside the outer portion (5), or
b. distal to the inner portion (2) and (b1) inside or (b2) outside the outer portion (5), or
c. inside the inner portion (2).

12. The device (1) according to claim 1, wherein any one of the inner portion (2), the suspension (3), the at least one rotation control device (4), the outer portion (5) or the at least one closing flap (8), if present, is made of at least one material selected from the group consisting of
a. a synthetic material,
b. a metal,
c. a magnetic or magnetisable material,
d. silicon, cardboard, or eva beads,
e. a glass, porcelain or ceramic substance,
f. wood or cork,
g. any combination of the above at least one material.

13. The device (1) according to claim 1, wherein the device (1) is a sanitary device, preferably a toilet rim block, an air modifying device, or a device for use within another device, a device for use in an open area, or an air refreshing device for use in a transportation vehicle.

14. The device (1) according to claim 1, wherein all of the compartments of the inner portion (2) are constructed
a. to be refillable, or
b. not to be refillable.

15. A device (1) according to claim 3, wherein the at least one ingredient is selected from the group consisting of
a. a fragrance;
b. a substance with anti-malodour activity;
c. a cleaning agent selected from sodium laureth sulfate, sodium dodecylbenzene sulfonate, fatty alcohol ethoxylate, oxoalcohol C13-15 ethoxylated, sodium lauryl sulphate or alkyl polyglycoside, an acid cleaner;
d. a foam former selected from sodium laureth sulfate, sodium dodecylbenzene sulfonate, fatty alcohol ethoxylate, oxoalcohol C13-15 ethoxylated, sodium lauryl sulphate or alkyl polyglycoside;
e. a chalk preventer selected from acids preferably being selected from the group consisting of citric acid, amidosulfonic acid, acetic acid, phosphoric acid, hydrochloric acid or lactic acid;
f. a complexing agent selected from tetrasodium EDTA, diethylenetriamine pentamethylene phosphonic acid sodium salt, tri-sodiumcitrate, methylglycinediacetic acid, etidronic acid, a polyphosphate or a phosphonate;
g. an oxidative agent selected from a peroxide or a hypochloride
h. an antibacterial agent selected from an alcohol, chlorine, hydrogen peroxide, a quaternary ammonium compound, sodium hypochlorite, chlorine dioxide, a chloramine, a copper or silver ion, or colloidal silver, or bromine; or
i. any mixture or combination of the above at least one ingredient.

16. The device (1) according to claim 4, wherein the outer portion (5) has one or more opening(s) (7).

17. The device (1) according to claim 11, wherein the one or more rotation control device(s) (4) is/are positioned inside the inner portion (2).

18. The device (1) according to claim 12, wherein any one of the inner portion (2), the suspension (3), the at least one rotation control device (4), the outer portion (5) or the at least one closing flap (8), if present, is made of at least one material selected from the group consisting of:
a. a synthetic material selected from polyethylene terephthalate (PET), polyethylene terephthalate-G (PETG), polyvinyl chloride (PVC), polypropylene (PP), polystyrene (PS), high impact polystyrene (HIPS), polyvinylidene chloride (PVDC), high-density polyethylene (HDPE) or low-density polyethylene (LDPE),
b. a metal selected from such as copper, aluminium, rust free iron, steel,
c. a magnetic or magnetisable material,
d. silicon, cardboard, or eva beads,
e. a glass, porcelain or ceramic substance,
f. wood or cork, or
g. any combination of the above at least one material.

19. A method to modify or mask malodour in a variety of environments comprising:
providing a device of claim 3,
releasing at least one ingredient selected from the group consisting of:
a. a fragrance;
b. a substance with anti-malodour activity;
c. a cleaning agent;
d. a foam former;
e. a chalk preventer;
f. a complexing agent;
g. an oxidative agent;
h. an antibacterial agent; or
i. any mixture or combination of the above at least one ingredient.

20. The method of claim 19, wherein the at least one ingredient selected from the group consisting of:
a. a fragrance;
b. a substance with anti-malodour activity;
c. a cleaning agent selected from sodium laureth sulfate, sodium dodecylbenzene sulfonate, fatty alcohol ethoxylate, oxoalcohol C13-15 ethoxylated, sodium lauryl sulphate or alkyl polyglycoside, an acid cleaner;
d. a foam former selected from sodium laureth sulfate, sodium dodecylbenzene sulfonate, fatty alcohol ethoxylate, oxoalcohol C13-15 ethoxylated, sodium lauryl sulphate or alkyl polyglycoside;
e. a chalk preventer selected from preferably being selected from the group consisting of citric acid, amidosulfonic acid, acetic acid, phosphoric acid, hydrochloric acid or lactic acid;
f. a complexing agent selected from tetrasodium EDTA, diethylenetriamine pentamethylene phosphonic acid sodium salt, tri-sodiumcitrate, methylglycinediacetic acid, etidronic acid, a polyphosphate or a phosphonate;
g. an oxidative agent selected from peroxide or a hypochloride;
h. an antibacterial agent selected from an alcohol, chlorine, hydrogen peroxide, a quaternary ammonium compound, sodium hypochlorite, chlorine dioxide, a chloramine, a copper or silver ion, or colloidal silver, or bromine; or
i. any mixture or combination of the above at least one ingredient.
